(19) United States
(12) Patent Application Publication
(10) Pub. No.: US 2026/0335496 A1
(43) Pub. Date: Oct. 29, 2026

(54) COHESIVE SHEAR-THINNING BIOMATERIALS AND THE USE THEREOF

(71) Applicants: The Regents of the University of California, Oakland, CA (US); The Penn State Research Foundation, University Park, PA (US)

(72) Inventors: Amir Sheikhi, State College, PA (US); Avijit Baidya, Los Angeles, CA (US); Alireza Khademhosseini, Los Angeles, CA (US)

(73) Assignees: The Regents of the University of California, Oakland, CA (US); The Penn State Research Foundation, University Park, PA (US)

(21) Appl. No.: 18/994,885

(22) PCT Filed: Aug. 9, 2023
(86) PCT No.: PCT/US2023/071909

§ 371 Date: Jan. 15, 2025

Related U.S. Application Data
(60) Provisional application No. 63/396,367, filed on Aug. 9, 2022.

(51) Int. Cl. A61L 24/00; A61L 24/02; A61L 24/10

(52) U.S. Cl. CPC A61L 24/0031; A61L 24/0015; A61L 24/02; A61L 24/104; A61L 2400/12

(57) ABSTRACT

Shear-thinning hydrogels are suitable biomaterials for catheter-based minimally invasive therapies; however, the tradeoff between injectability and mechanical integrity has limited their applications, particularly at high external shear stress such as endovascular procedures. The invention provides an easily injectable, non-hemolytic, and non-cytotoxic shear-thinning hydrogel with significantly enhanced cohesion via controlling noncovalent interactions.

### CROSS REFERENCE TO RELATED APPLICATIONS

This application claims the benefit under 35 U.S.C. Section 119(e) of co-pending and commonly-assigned U.S. Provisional Patent Application No. 63/396,367, filed Aug. 9, 2022, entitled “COHESIVE SHEAR-THINNING BIOMATERIALS AND THE USE THEREOF”, which application is incorporated by reference herein.

### STATEMENT REGARDING FEDERALLY SPONSORED RESEARCH AND DEVELOPMENT

This invention was made with government support under HL140951, awarded by the National Institutes of Health. The government has certain rights in the invention.

### TECHNICAL FIELD

The present invention relates to shear thinning biomaterials and methods for making and using them.

### BACKGROUND OF THE INVENTION

Shear-thinning hydrogels are non-Newtonian materials that behave as viscous fluids under shear stress and then recover solid-like properties upon elimination of the stress. Due to these properties, injectable shear-thinning biomaterials (STB) are attracting attention as a group of self-healing materials that allow for fluent infusion and local equilibrium after approaching the final application site. In clinical applications, STBs can be delivered into the body using a needle or a general/microcatheters by manual pressure. To optimize the clinical application, it is necessary to adjust the physical properties of STB according to the specific clinical situations. By changing the physical properties, one can synthesize tailored hydrogels for specific clinical applications, such as embolizing a certain size of blood vessel, controlled drug release, and modulation of the stiffness of tissue engineering scaffolds.

The physical properties of conventional STBs can be modulated by a combination of several carbon-based, polymeric, and inorganic nanomaterials. Several biomaterials, such as gelatin, hyaluronic acid, chitosan, collagen, and alginate have been previously used along with inorganic constituents to form STBs. In particular, gelatin limits the adsorption of nonspecific proteins, enhanced hemolysis, and ultimately prolongs clotting time, demonstrating substantially improved hemocompatibility of STB in vitro. Furthermore, the application of gelatin in tissue engineering and regenerative medicine has been approved by the Food and Drug Administration (FDA). Conventional STBs are prepared by mixing gelatin with synthetic clay nanoparticles, Laponite®, for hemostasis and endovascular embolization. These STBs exhibit strong shear-thinning behavior as well as biocompatible properties ranging from blood coagulation to minimized inflammatory response. Others have extended this work to implement STBs as embolic agents, functionalized scaffolds, 3D-bioinks and drug delivery systems. Unfortunately, however, synthetic clay nanoparticles such as Laponite are crystallized nanoparticles and the size, surface chemistry of such materials are not easily tuned.

For the reasons noted above, there is a need in the art for new shear-thinning materials and methods for making and using them.

### SUMMARY OF THE INVENTION

Shear-thinning hydrogels are highly desirable biomaterials for catheter-based minimally invasive therapies. Unfortunately however, the tradeoff between injectability and mechanical integrity has limited their applications, particularly at high external shear stress such as endovascular procedures. To address this material design challenge, we developed an easily injectable, non-hemolytic, and non-cytotoxic shear-thinning hydrogel with significantly enhanced cohesion via controlling noncovalent interactions.

Embodiments of the invention include, for example, shear-thinning biocompatible compositions of matter comprising silicate nanoparticles or nanoplatelets, one or more cationic polymers such as Polydiallyldimethylammonium chloride, and gelatin. While Polydiallyldimethylammonium chloride is used as an exemplary cationic polymer in the illustrative working embodiments of the invention that are disclosed herein, embodiments of the invention can utilize other cationic polymers such as Poly(2-dimethylamino)ethyl methacrylate) methyl chloride quatemary salt, Poly[(2-ethyldimethylammonioethyl methacrylate ethyl sulfate)-co-(1-vinylpyrrolidone)], Poly(acrylamide-co-diallyldimethylammonium chloride) and the like. In addition, artisans can utilize a wide variety of cationic monomers to make cationic polymers useful in embodiments of the invention such as Diallyldimethylammonium chloride monomers, [2-(Methacryloyloxy)ethyl]trimethylammonium chloride monomers, [2-(Acryloyloxy)ethyl]trimethylammonium chloride monomers, (3-Acrylamidopropyl)trimethylammonium chloride monomers, [3-(Methacryloylamino)propyl]trimethylammonium chloride monomers and the like. As is known in the art, cationic polymers can be formed using a single type of cationic monomer or mixture of different cationic monomers.

In certain embodiments of the invention, the constituents or relative amounts the constituents are selected to tune or modulate one or more properties of the composition. For example, in typical embodiments of the invention, the silicate nanoparticles or nanoplatelets have a median diameter of from 5 nm to 150 nm and/or comprise a negative charge at physiological pH. Optionally the silicate nanoparticles or nanoplatelets comprise from 5% to 45% (w/v) of the composition; and/or the cationic polymer comprises from 1% to 10% (w/w); and/or the gelatin comprises from 1% to 30% of the composition. In certain embodiments of the invention, the silicate nanoparticles or nanoplatelets comprise not more than 9% (w/v) of the composition; and/or the cationic polymer comprises at least 1, 2, 3, 4 or 5% (w/w) of the composition; and/or the cationic polymer comprises less than 10, 9, 8, 7 or 6% (w/w) of the composition. In some embodiments of the invention, the composition is disposed within a vessel (e.g., a catheter) selected for its ability to facilitate a user modulating one or more rheological properties of the composition.

Certain embodiments of the compositions of the invention include additional agents such as a pharmaceutical excipient selected from the group consisting of a preservative, a tonicity adjusting agent, a detergent, a viscosity adjusting agent, a sugar and a pH adjusting agent. Optionally, the compositions of the invention include one or more therapeutic agents such as an anti-inflammatory agent, an agent that modulates coagulation, an antibiotic agent, a chemotherapeutic agent or the like.

Another embodiment of the invention is a method of making a shear-thinning biocompatible composition disclosed herein comprising combining together silicate nanoparticles or nanoplatelets, a cationic polymer and gelatin, and optionally a pharmaceutical excipient and/or a therapeutic agent so as to form a shear-thinning biocompatible composition. In certain embodiments of these methods, a surface property of the silicate nanoparticles or nanoplatelets, a median diameter of the silicate nanoparticles or nanoplatelets, a relative amount of silicate nanoparticles or nanoplatelets; and/or a relative amount of cationic polymer, gelatin or the like is selected to tune or modulate one or more rheological properties of the shear-thinning biocompatible composition. By modulating the mechanical properties of the compositions of the invention in this manner, embodiments of the invention can be tailored for use in a variety of different clinical applications.

Yet another embodiment of the invention is a method of delivering a shear-thinning biocompatible composition disclosed herein to a preselected site (e.g. an in vivo location where an individual has experienced trauma or injury). Typically, such methods comprise disposing the composition in a vessel having a first end comprising an opening and a second end (e.g. a catheter); applying a force to the second end of the vessel, wherein the force is sufficient to liquify the composition; and then delivering the composition out of the vessel through the opening and to the preselected site.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: Developing cohesive shear-thinning biomaterials. (a) Schematic of pGL biomaterial preparation. (b) Key molecular interactions between the building blocks of pGL biomaterial, including gelatin, LAPONITE, and PDDA. (c) Images of (i) pGL biomaterial deployed from a 5F catheter in air, and the stability of pGL biomaterial in (ii) milli-Q water and (iii) DPBS. (d) Schematic of the interactions among the building blocks of GL biomaterial as PDDA concentration increases with insets showing the physical appearance of biomaterials.

FIG. 2: Optimization and characterization of shear-thinning biomaterials. (a) Injection force of the GL biomaterials containing varying concentrations of PDDA through a 5F catheter at an injection rate of 2 mL min⁻¹. (b) ζ-potential of gelatin-LAPONITE aggregates containing varying concentrations of PDDA. Based on the biomaterial design criteria for endovascular procedures, i.e., low injection force, homogeneity, and cohesion, the pGL biomaterial (GL containing 5% w/w of PDDA, shown in the purple shade) is selected for further characterization and comparison with the GL biomaterial (shown in the gray shade). (c) Injection force of pGL biomaterial through different syringes with a 5F catheter at an injection rate of 2 mL min⁻¹. (d) Force required to inject pGL through catheters with varying diameter using a 3 ml syringe at an injection rate of 2 mL min⁻¹. (e) Storage modulus (G′) and loss modulus (G″) of pGL and GL biomaterials versus shear strain. Cyclic (f) storage modulus and (g) loss modulus recovery of pGL and GL biomaterials at high (100%) and low (1%) oscillatory strain rates. (h) Viscosity versus steady shear rate for the pGL and GL biomaterials. (i) Shear stress versus shear rate for the pGL and GL biomaterials.

FIG. 3: Cohesive shear-thinning biomaterial successfully occludes in vitro aneurysm models. (a) Schematic of a side-wall large-neck aneurysm model and (b) its corresponding experimental setup loaded with the biomaterial. (c) Quantification of material loss under constant fluid flow rates after 24 h. (d) Schematic representation of the bifurcation aneurysm model, loaded with the biomaterial using a clinically relevant 5F catheter. (e) Corresponding experimental setup for the bifurcation aneurysm model before (i) and after (ii) loading with the pGL biomaterial.

FIG. 4: Cohesive shear-thinning biomaterial enables in-flow occlusion of patient-derived intracranial aneurysm model in vitro. (a) Structure of the patient-derived basilar tip (intracranial) aneurysm site from different angles and (b) image of the fabricated aneurysm PDMS models before (i) and during (ii) catheter-assisted loading of pGL biomaterial. Red dye was used to enhance the contrast of biomaterial with the clear liquid and PDMS. (c) In-flow injection (i) and retention (iii) of the pGL biomaterial in the patient derived aneurysm model compared with the injection (ii) and lack of retention (iv) of GL biomaterial. Inset of iv shows the loss of GL material during delivery as a result of fragmentation. (d) Success rate of GL and pGL biomaterials delivery under the carotid flow, defined as the number of successful delivery attempts (without fragmentation) to the total number of attempts (n=20).

FIG. 5. Cytotoxicity and hemocompatibility of the shear-thinning biomaterials. (a) Representative live/dead assay fluorescence images of HUVECs incubated for 4 days in the absence (control) and presence of the GL or pGL biomaterials. Quantification of the (b) NIH/3T3 fibroblast cell and (c) HUVEC viability, calculated from the live/dead image analysis on day 4 of culture. Fluorescence intensity recorded from the PrestoBlue™ assay on days 1 and 4 of culture for the (d) NIH/3T3 fibroblast cells and (e) HUVECs in the absence (control) and presence of the biomaterials (GL or pGL). (f) Images of the hemolysis assay conducted using the biomaterials (GL and pGL) compared with the positive control (PC; PEG) and negative control (NC; Triton X-100). (g) Quantification of RBC hemolysis percentage after incubation with the biomaterials compared with the control groups.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Shear-thinning hydrogels are suitable biomaterials for catheter-based minimally invasive therapies; however, the tradeoff between injectability and mechanical integrity has limited their applications, particularly at high external shear stress such as endovascular procedures. Extensive molecular crosslinking often results in stiff, hard-to-inject hydrogels that may block catheters, whereas weak crosslinking renders hydrogels mechanically weak and susceptible to shear-induced fragmentation. Thus, controlling molecular interactions is necessary to improve the cohesion of catheter-deployable hydrogels. To address this persistent material design challenge, we have developed an easily injectable, non-hemolytic, and non-cytotoxic shear-thinning hydrogel with significantly enhanced cohesion via controlling noncovalent interactions. We show that enhancing the electrostatic interactions between weakly bound biopolymers (gelatin) and nanoparticles (silicate nanoplatelets) using a highly charged polycation at an optimum concentration increases cohesion without compromising injectability, whereas introducing excessive charge to the system leads to phase separation and loss of function. The cohesive biomaterial is successfully injected with a neuroendovascular catheter and retained without fragmentation in patient-derived three-dimensionally (3D) printed cerebral aneurysm models under a physiologically relevant pulsatile fluid flow, which would otherwise be impossible using the non-cohesive hydrogel counterpart. This work sheds light on how charge-driven molecular and colloidal interactions in shear-thinning physical hydrogels improves cohesion, enabling complex minimally invasive procedures under flow, which may open new opportunities for developing the next generation of injectable biomaterials.

As noted above, embodiments of the invention include biocompatible compositions of matter comprising silicate nanoparticles or nanoplatelets, a cationic polymer such as Polydiallyldimethylammonium chloride, and gelatin. The compositions of the invention can include further constituents such as additional polymers, excipients, therapeutic agents and the like. For example, compositions of the invention can include one or more Food and Drug Administration (FDA) approved or cytocompatible polymers. Such polymers include alginate, chitosan, collagen, hyaluronic acid (HA), chondroitin sulfate (ChS), dextrin, gelatin, fibrin, peptide, and silk. Synthetic polymers such as poly(ethylene glycol) (PEG), poly(ethylene oxide) (PEO), poloxamer (Pluronic®) (PEO-PPO-PEO), polyoxamine (Tetronic®) (PEO-PPO), poly(vinyl alcohol) (PVA), poly(lactic-co-glycolic acid) (PLGA), poly(glycolic acid) (PGA), poly(lactic acid) (PLA), polycaprolactone (PCL), poly(L-glutamic acid) (PLga), polyanhydrides, poly(N-isopropylacrylamide) (PNIPAAm), polyaniline and the like can also be included in compositions of the invention. As is known in the art, preparations of hydrogels can be made to include either chemically or physically crosslinked materials.

In illustrative embodiments of the invention, the nanoparticles or nanoplatelets comprise Laponite®, a synthetic smectite clay that has a number of technological applications. In biomedical applications, particularly in nanomedicine, this material holds great potential. Laponite® is a 2-dimensional (2D) nanomaterial composed of disk-shaped nanoscale crystals that have a high aspect ratio. Certain embodiments of the compositions of the invention include, for example nanoparticles or nanoplatelets combined with a pharmaceutical excipient such as one selected from the group consisting of a preservative, a tonicity adjusting agent, a detergent, a viscosity adjusting agent, a sugar and a pH adjusting agent. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

Optionally, the compositions of the invention include one or more therapeutic agents such as an anti-inflammatory agent, an agent that modulates coagulation, an antibiotic agent, a chemotherapeutic agent or the like. Compositions of the invention can be formulated for use as carriers or scaffolds of therapeutic agents such as drugs, cells, proteins, and bioactive molecules (e.g., enzyme). As carriers, such compositions can incorporate the agents and deliver them to a desired site in the body for the treatments of a variety of pathological conditions. These include, for example, infectious and inflammatory diseases (e.g. Parkinson's disease, bacterial and antimicrobial infection, diabetes and the like) as well as cancers (e.g. colon, lung, breast, ovarian, lymphoma cancers and the like). In addition, as scaffolds, compositions of the invention can provide a flexible dwelling space for cells and other agents for use in tissue repair and the regeneration of desired tissues (e.g. for cartilage, bone, retina, brain, and, neural tissue repair, vascular regeneration, wound healing and the like). Moreover, embodiments of the invention can include immunomodulatory agents useful for immunotherapy in order to, for example, enhance components of the immune system. Certain illustrative materials and methods that can be adapted for use in such embodiments of the invention are found, for example in Hydrogels: Design, Synthesis and Application in Drug Delivery and Regenerative Medicine 1st Edition, Singh, Laverty and Donnelly Eds; and Hydrogels in Biology and Medicine (Polymer Science and Technology) UK ed. Edition by J. Michalek et al.

As noted above, embodiments of the invention include methods of making a shear-thinning biocompatible composition disclosed herein comprising combining together silicate nanoparticles or nanoplatelets, a cationic polymer and gelatin, and optionally a pharmaceutical excipient and/or a therapeutic agent so as to form a shear-thinning biocompatible composition. In certain embodiments of these methods, a surface property of the silicate nanoparticles or nanoplatelets, a median diameter of the silicate nanoparticles or nanoplatelets, a relative amount of silicate nanoparticles or nanoplatelets; and/or a relative amount of cationic polymer, gelatin or the like is selected to tune or modulate one or more rheological properties of the shear-thinning biocompatible composition. By modulating the mechanical properties of the compositions of the invention in this manner, embodiments of the invention can be tailored for use in a variety of different clinical applications.

In certain embodiments of the methods of making a shear-thinning biocompatible composition the method is selected to form a shear-thinning biocompatible composition exhibiting an injection force of less than 10 Newtons when extruded through a 5F catheter at an injection rate of 2 mL min⁻¹ under physiological conditions. In certain embodiments of the methods of making a shear-thinning biocompatible composition, the method is selected to form a shear-thinning biocompatible composition exhibiting ζ-potential from −30 millivolts to −40 millivolts under physiological conditions. In typical embodiments of the methods of making a shear-thinning biocompatible composition, the silicate nanoparticles or nanoplatelets are selected to have a median diameter of from 5 nm to 150 nm; and/or the silicate nanoparticles or nanoplatelets are selected to comprise from 5% to 45% (w/w) of the composition; and/or the cationic polymer concentration in gelatin is selected to comprise at least 1, 2, 3, 4 or 5% (w/w); and/or the cationic polymer concentration in gelatin solution is selected to comprise less than 10, 9, 8, 7 or 6% (w/w); and/or the gelatin is selected to comprise from 1% to 30% of the composition. In certain illustrative embodiments of the invention, the silicate nanoparticles or nanoplatelets comprise not more than 9% (w/v) of the composition; and the gelatin comprises not more than 18% (w/v) of the composition. Optionally, the methods combine the silicate nanoparticles or nanoplatelets with a pharmaceutical excipient and/or a therapeutic agent.

Another embodiment of the invention is a method of delivering a shear-thinning biocompatible composition disclosed herein to a preselected site (e.g. an in vivo location where an individual has experienced trauma or injury). Typically, such methods comprise disposing the composition in a vessel having a first end comprising an opening and a second end (e.g. a catheter); applying a force to the second end of the vessel, wherein the force is sufficient to liquify the composition; and then delivering the composition out of the vessel through the opening and to the preselected site.

Certain aspects of the invention are discussed in Baidya et al., ACS Appl Mater Interfaces. 2022 Sep. 28;14(38):42852-42863; Epub 2022 Sep. 19 (hereinafter “Baidya et al.”, the contents of which are incorporated by reference.

### Illustrative Materials and Methods of the Invention

Hydrogels with multifunctional macroscopic functionalities have enabled a broad spectrum of advanced medical therapeutics¹⁻⁵. The physical properties of hydrogels such as injectability and mechanical robustness are regulated by the molecular interactions among their building blocks, such as polymer chains and nanoparticles via noncovalent and/or covalent binding within a three-dimensional (3D) network⁶⁻⁹. For example, covalent bonds impart mechanical resilience to elastic hydrogel biomaterials, whereas noncovalent interactions, such as ionic binding, often enable energy dissipation to withstand cyclic deformations and minimize mechanical mismatch at soft tissue interfaces¹⁰⁻¹¹.

To enable minimally invasive therapeutics, various injectable biomaterials have been developed that form gels immediately after injection¹²⁻¹³. This gel formation mechanism is favorable in minimally invasive procedures, such as endovascular embolization or aneurysm treatment;¹⁴ however, delays in biomaterial crosslinking and lack of cohesion may cause material loss in body fluids, e.g., blood, and block blood vessels¹⁵. Moreover, fast crosslinking may block the injection tools such as catheters or needles during the operation, endangering patients' life^{3, 15}. In general, although time-dependent crosslinking mechanisms may enable hydrogels to readily pass through needles, they often face major challenges with injection through long surgical catheters³. Therefore, there is typically a tradeoff between injectability and mechanical integrity: extensive chemical crosslinking often limits catheter-injectability, whereas weak crosslinking renders hydrogels mechanically naive and susceptible to shear-induced fragmentation³.

To overcome the poor catheter injectability of covalently crosslinked hydrogels, shear-thinning biomaterials have been developed based on reversibly deformable molecular and/or colloidal networks¹⁴. Mechanistically, these materials with non-Newtonian characteristics undergo spontaneous molecular rearrangements under shear^{3, 15-16}, facilitating injection. As an example, nanosilicate-based shear-thinning hydrogels have been synthesized to occlude arteries and veins¹⁷ through endovascular techniques. Injectability and stability of these hydrogels rely on noncovalent interactions among their components, which regulate their flow under shear and gel formation upon shear elimination, e.g., after injection¹⁷⁻¹⁸.

Although these shear-thinning biomaterials have suitable catheter-injectability, they face severe challenges in terms of their mechanical stability (robustness) under physiological fluid flow conditions. This was clearly observed when a gelatin-based shear-thinning material was used to occlude a berry type, neck-less aneurysm model under physiologically relevant fluid flows¹⁹. Although the biomaterial was previously proven to be successful for endovascular embolization¹⁷, direct fluid flow permanently disrupted the hydrogel network, causing disintegration and fragmentation. Therefore, increasing the cohesion of shear-thinning hydrogels without compromising their injectability may improve their stability and integrity for the minimally invasive treatment of medical complications, such as cerebral aneurysms and cerebrospinal fluid (CSF) leak. In general, the treatment of fragile cerebral aneurysms is highly challenging²⁴ and can cause fatal brain stroke upon rupture²¹. Current treatment procedures with microsurgical clipping²² or coiling²³⁻²⁴ are limited due to the risks of coil detachment, vasospasm²⁵, poor reendothelialization¹, and coil migration^{2,3}. Liquid/gel embolic materials that solidify upon injection in aqueous media are also limited in efficacy as they increase the risk of spilling and catheter entrapment/cementing²⁵. Onyx, one of such embolic materials, has already been used for the occlusion of aneurysms²⁶. Meanwhile, organic solvents in embolic materials, such as dimethyl sulfoxide (DMSO), may result in systemic cardiovascular toxicity and vasospasm²⁷⁻²⁸. Accordingly, designing a catheter-injectable shear-thinning biomaterial with controlled molecular/colloidal interactions, specifically engineered to maximize cohesion may open new opportunities for the treatment of potentially fatal conditions, such as aneurysms.

Here, we aim to develop a cohesive shear-thinning biomaterial via regulating reversible electrostatic interactions while preserving the viscoelastic properties and catheter injectability. Our hypothesis is that strengthening electrostatic attractions between gelatin and nanosilicates using a highly cationic polymer such as PDDA without inducing phase separation will improve hydrogel cohesion while preserving the shear-thinning properties. The physical properties and occlusion capability of engineered cohesive shear-thinning hydrogel in 3D-printed standard or complex patient-derived in vitro aneurysms models will be characterized. As biomaterial-enable endovascular procedures involve direct material contact with blood and blood vessels, the hemolytic properties and in vitro cytotoxicity of hydrogel will also be assessed to set the stage for future in vivo studies.

### MATERIALS AND METHODS

### Materials

Gelatin (type A, from porcine skin, gel strength ~300 g Bloom), poly(diallyldimethylammonium chloride) 20% wt. in water (PDDA), hemoglobin human, lyophilized powder, Drabkin's reagent, poly(ethylene glycol) (PEG, Mn=400), acetone, and Triton X-100 were purchased from Sigma-Aldrich (Milwaukee, WI, USA). LAPONITE® XLG-XR (synthetic silicate-based nanoplatelets) was purchased from Southern Clay Products, Inc. (Louisville, KY, USA). Polydimethylsiloxane (PDMS) was obtained from Ellsworth Adhesives (Irvine, CA, USA) and used to make in vitro aneurysm models. Commercially available food-grade red dye was purchased from local store to improve the contrast of the images. NIH/3T3 murine fibroblasts (ATCC®) CRL-1658™), and human umbilical vein endothelial cells (HUVEC, ATCC® CRL-1730™) were procured from ATCC (VA, USA). Dulbecco's Phosphate-buffered saline (DPBS, 1X) used in the flow experiments and cell culture study was obtained from Fisher Scientific (Hampton, NH, USA). PrestoBlue™ cell viability reagent and Live/Dead viability/cytotoxicity kit, for mammalian cells were purchased from Invitrogen (NY, USA). Dulbecco's modified Eagle's medium (DMEM, fetal bovine albumin (FBS), qualified, heat inactivated, Penicillin/Streptomycin (10,000 U/mL), trypsin-EDTA phenol red (0.25%, 1X) were bought from Gibco (NY, USA). Endothelial basal medium (EBM-2) and endothelial growth BulletKit were obtained from Lonza (Basel, Switzerland).

### Methods

### Biomaterial Preparation

The shear-thinning biomaterials were prepared based on our established protocol¹⁷ with further modifications to increase the cohesion. An aqueous gelatin solution (18% w/v) at 37 □C was homogeneously mixed with PDDA solution at varying concentrations (0, 1, 2, 3, 5, 7, and 10% w/w). In parallel, a 9% w/v LAPONITE gel was prepared via the exfoliation of LAPONITE in cold milliQ water (4 °C) using vigorous vortexing for at least 10 min, followed by mixing with the gelatin/PDDA polymer solutions and vortexing with intermittent spatula-assisted shearing. To ensure the homogeneity of PDDA-incorporated biomaterial, mixing was continued using a speed mixture for at least 5 min at 3000 rpm. The total solid mass content of biomaterials was maintained at 6% w/v. As a control, gelatin-LAPONITE biomaterial (GL) was prepared using the same method without including PDDA. The GL containing the optimum concentration of PDDA (5% w/w) had the lowest injection force and highest cohesion. This biomaterial is called pGL as it is made up of an optimum PDDA (p) concentration, gelatin (G), and LAPONITE (L).

### Injection Force Measurements

The injectability of biomaterials was characterized by injection force measurements using an Instron Universal Testing System (Model 5943). Several parameters pertaining to biomaterial injectability, including syringe volume and catheter diameter, were investigated to evaluate the applicability of the biomaterials in minimally invasive, catheter-based procedures. Syringes (BD Biosciences) loaded with the biomaterials (pGL or GL) were attached to medical catheters (Cook medical) and mounted in the material testing system using a tension grip around the luer lock connecting port of catheter. A compressive plate depressed the syringe plunger at a constant rate of 2 mL min⁻¹, and the material testing system was used to measure the force on the plate over time using the Bluchill® universal software (version 3). The injection force was recorded as the load (N) when the injection force reached a plateau.

### Effect of Catheter Diameter on Biomaterial Injectability

To understand flowability of the engineered biomaterials through catheters, different catheter diameters relevant to neuroendovascular treatments, including 6F, 5F and 3.3F (length=100 cm), were used to measure the injection force, while other parameters were maintained unchanged.

### Effect of Syringe Volume on Biomaterial Injectability

To investigate the effect of syringe volume on the injection force, varying syringes (1 mL, 3 mL, and 5 mL) were used in combination with the 5F catheter to measure the injection force of biomaterials at a constant depression rate of 33.96 mm min⁻¹ (2 mL min⁻¹).

### Effect of Flow Rate on Biomaterial Injectability

Different flow rates were investigated in combination with the standard 3 ml syringe and 5F catheter. This was conducted using different depression rates, including~1 mL min⁻¹, 2 mL min⁻¹, or 3 mL min⁻¹.

### 2.2.3. Rheological Assessments

The rheological properties of biomaterials were characterized using an Anton Paar Rheometer (model MCR 302). The storage modulus (G′) and loss modulus (G″) were measured using a parallel plate geometry with a truncation gap of 1 mm. Samples (~750 μL) were loaded to the sandblasted plate (diameter=20 mm) and allowed to equilibrate for 90 s. Oscillatory strain sweeps were conducted at a range of 0.01-100% and a constant angular frequency of ~10 rad s⁻¹, and oscillatory angular frequency sweeps were performed at 0.1-100 rad s⁻¹ and a constant stain of 0.1% at 25 °C. Viscosity was measured based on steady shear rheology at shear rates ranging from 0.01 to 10 s⁻¹ at 25 °C.

### ζ-Potential Measurement

As the electrostatic interactions of charged LAPONITE nanosilicates with gelatin and many other charged polymers are well investigated²⁹⁻³⁰, here, we only measure the ζ-potential of LAPONITE-gelatin aggregates at varying concentrations of added PDDA. For this, hydrogels were synthesized with varying PDDA concentrations, disintegrated in water, and the ζ-potential of aggregates was measured using a previously established protocol with further modifications³¹. Briefly, as-prepared hydrogels (10 mg) were dispersed in Milli-Q water (10 mL) by sonication and vortexing for 2 h, and the ζ-potential of the aggregates was measured at 25 °C in at least triplicates with 20 scans using Zetasizer Nano series (Malvern Instruments).

### Fabrication of In Vitro Aneurysm Models

To test the biomaterial stability in aneurysms, sessile (neck-free) aneurysm PDMS models were designed, and 3D printed in two parts, including a top and a bottom portion, enabling facile dismantling. The top portion consisted of an aneurysm sac hemisphere with a diameter of 6 mm. The bottom portion consisted of a cylindrical blood vessel with a diameter of 6 mm and a length of 75 mm, mimicking small- or medium-sized saccular cerebral aneurysms³². When assembled, the hemisphere on the top portion was attached to the bottom portion, making a closed system, mimicking that of an aneurysm-affected blood vessel. The negative molds consisting of the (i) hemispheres and (ii) blood vessels were fabricated by 3D printing of acrylonitrile butadiene styrene (ABS) filaments using a Lulzbot (info) instrument. Next, these pieces were glued into custom-built laser-cut acrylic boxes, followed by filling with PDMS and curing in a hot air oven at 80 °C for 2 h. The cured PDMS was removed from the acrylic molds and the ABS was dissolved using acetone. For the flow experiments, completed models consisting of a top portion (hemisphere) and bottom portion (blood vessels) were assembled between two acrylic plates, bolted together to hold and seal the two pieces of aneurysm model. A similar procedure was followed to prepare the bifurcation model, which contained a cylindrical blood vessel with a diameter of 5 mm. In this case, the diameter of aneurysm sac was 9 mm.

### Biomaterial Retention Quantification in Aneurysm Models In Vitro

The retention (stability) of biomaterials was investigated by filling the aneurysm models with GL or pGL, and exposing them to constant DPBS flow for 24 h, followed by mass loss quantification. The top hemisphere of each aneurysm model was filled with the biomaterials using a ImL syringe attached to the 5F catheter, which was then assembled to the bottom section prior to flow experiments. The biomaterial-loaded aneurysm sac (top section) was weighed to obtain the initial biomaterial mass. Next, the assembled model was exposed to 15 or 20 ml s⁻¹ constant flow of DPBS. After 24 h, the flow was stopped, and the biomaterial was removed and freeze-dried. To quantify the mass loss, the dry weight of samples after flow experiments were compared with the dry weight of initial biomaterial placed in the model before the flow experiments.

### Fabrication of a Patient-Derived Aneurysm Model and Filling It Under Pulsatile Flow (Carotid Flow)

The performance of pGL and GL biomaterials was compared with each other inside saccular aneurysms, specifically a patient-derived intracranial aneurysm under carotid flow. The basilar tip aneurysm model was created from human aneurysm 3D images using a previously developed method³³. Briefly, the positive mold made of ABS was fabricated using a 3D printer, and the surface of printed objects was smoothed by dipping in the ABS solvent (eSolve). The vascular mold was dried and coated with PDMS, followed by curing at 60 °C. The ABS mold was then removed by immersion in acetone³³. As a physiologically-relevant proof-of-concept, the biomaterials were injected in the patient-derived aneurysm model using a 4F catheter under the pulsatile (carotid) flow of DPBS at 4 mL s⁻¹ resembling the average blood flow in the basilar artery³⁴⁻³⁶. Images were acquired to investigate the occlusion of artery and the integrity (retention) of biomaterials.

### Cytotoxicity Assessments

Biomaterial cytotoxicity was evaluated using two different cell lines: NIH/3T3 murine fibroblasts and HUVECs. Fibroblast cells were cultured in DMEM supplemented with 10% (v/v) FBS and 1% (v/v) penicillin/streptomycin. HUVECs were cultured in EBM-2 and supplemented with endothelial growth BulletKit and 1% (v/v) penicillin/streptomycin. Cells were cultured at 37 °C in a 5% CO₂ incubator (Forma incubators, ThermoFisher Scientific, USA), and the media was changed three times a week until a confluency of ~90% was reached to used them for the experiments. The confluent cells were trypsinized using trypsin-EDTA (0.25%) and counted using a hemocytometer. NIH/3T3 (2000 cells/well) and HUVECs (5000 cells/well) were seeded in a separate 12-well plate and placed at 37 °C in a CO₂ incubator. After 24 h, 0.1 mL of ultraviolet (UV) sterilized (wavelength~250 nm, duration~1 h) biomaterials was injected at the side of each well using a needle-free 1 ml syringe and was incubated with the cells for 1 and 4 days. Thereafter, the PrestoBlue™ cell viability assay was conducted following the manufacturer's instructions to evaluate the metabolic activity of cells in contact with the biomaterials. Briefly, 1 mL of PrestoBlue™ reagent (10% in the complete medium) was added to each well in the dark and incubated at 37 °C for 1.5 h. Next, 100 μL of the supernatant from each well was transferred to a 96-well plate and the fluorescence was measured using a microplate reader (BioTek UV/vis Synergy 2, VT, USA) at excitation/emission wavelengths of 530/590 nm. The measurements were conducted for 4 replicates per sample and the cells not exposed to the biomaterials were used as a control. After completing a PrestoBlue™ assay on day 4, the cells were thoroughly washed with DPBS and 1 mL of calcein-AM/ethidium homodimer-1 Live/Dead viability/cytotoxicity assay at a concentration of 2 μM/4 μM was added to each well, and the cells were incubated in the dark for 15 min. The stained cells were imaged for live cells (Calcein-AM, green fluorescent excitation/emission 495 nm/515 nm) and dead cells (ethidium homodimer-1, red fluorescent excitation/emission 495 nm/635 nm) using an inverted fluorescence microscope (Axio Observer 5, Zeiss, Germany). The cell viability was quantified from 5 randomly selected areas of each well using ImageJ software³⁷ (Version 1.52e, USA).

### Hemolysis Assessment

To assess the hemolytic effects of biomaterials, a hemolysis assay was conducted following the ASTM E2524-08 standard.³⁸ Heparinized whole human blood was purchased from Zenbio (NC, USA). The concentration of the hemoglobin in the blood was calculated from the human hemoglobin standard curve using Drabkin's reagent. The blood was diluted with DPBS to rectify hemoglobin concentration to 10±2 mg/mL. The biomaterials were prepared as explained before and sterilized via UV exposure (wavelength=250 nm) for 1 h. Subsequently, 0.1 mL of the biomaterials was transferred to Eppendorf tubes containing 800 μL of DPBS and 100 μL of diluted blood. Similarly, positive and negative controls were prepared by adding 0.1 mL of PEG (final concentration of 4.4% v/v) and Triton X-100 (final concentration of 1% v/v) to the tubes, respectively. The biomaterial samples (0.1 mL) in 900 μL of DPBS were used as a blood-free control to identify possible false-positive assay results. All the samples were incubated at 37 °C for 3 h±15 min, followed by centrifugation for 15 min at 14000 rpm. Subsequently, 100 μL of supernatant was transferred to a 96-well plate and an equal volume of Drabkin's reagent was added to each well and allowed to react on a shaker for 15 min in the dark. The absorbance against the reagent was measured at 540 nm using a microplate reader, and the hemoglobin concentration in each sample was calculated from the standard curve. Hemolysis (%) was measured using the following formula:³⁹

Hemolysis (%)=Sample hemoglobin×100/diluted blood hemoglobin (~10 mg/mL)

### Statistical Analysis

Data analysis for the cell studies was carried out using a 1- or 2-way ANOVA test with GraphPad Prism 9 software. Error bars represent mean±standard deviation (SD) of measurements (*p<0.05, **p<0.01, ***p<0.001, and ****p<0.0001).

### Results and Discussion

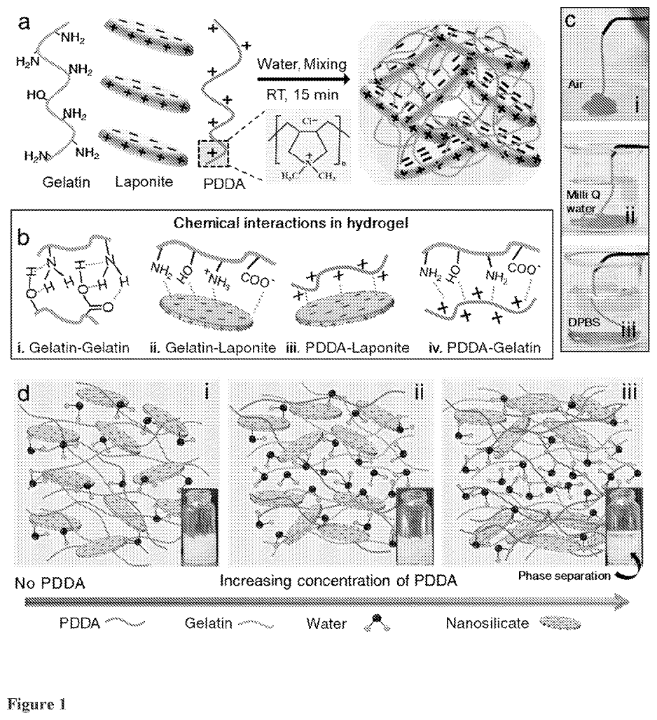

FIG. 1a schematically represents the preparation of cohesive shear-thinning biomaterial (i.e., pGL), comprising biocompatible and biodegradable LAPONITE nanoplatelets²⁹, gelatin, and PDDA. LAPONITE nanoplatelets are decorated with negative and positive charges on the surface and edge (rim), respectively, which interact with gelatin and many other biopolymers used for diverse biomedical applications^{17, 29, 40}. However, modes of intermolecular interactions between LAPONITE and gelatin are limited because of their predominant negative charges,⁴¹ weakening the biomaterial cohesion. In addition, controlling the dipolar interactions in LAPONITE-based hydrogel networks is challenging as the nanoplatelets readily form aggregates through electrostatic attractions with positively charged moieties/polymers and often disrupt the homogeneity of the biomaterial, leading to phase separation²⁹. Accordingly, the pGL biomaterials were engineered by regulating the dipolar interactions though solvation-induced charge dilution, a well-known mechanism in peptide based therapeutics⁴². We hypothesize that the incorporation of positively charged PDDA in gelatin solution, followed by mixing with LAPONITE controls the electrostatic attraction of polymers with the anionic surface of LAPONITE nanoplatelets, reducing the net anionic group density of composite hydrogel (gelatin at the intrinsic pH of LAPONITE dispersion, i.e., pH=9-10). Thus, the extended attraction between the LAPONITE and polymers may improve the cohesion of the shear-thinning hydrogel, while maintaining the homogeneity and injectability.

While gelatin chains bearing pH-induced charged groups (e.g., carboxylate groups, —COO⁻ at pH>9) possess limited electrostatic interactions with LAPONITE, increasing the positive charge density via adding PDDA may facilitate the formation of extended noncovalent interactions, such as electrostatic attraction and possibly hydrogen bonding (FIG. 1b). The properties of GL hydrogel are regulated by gelatin-gelatin and gelatin-LAPONITE interactions, which are usually weak⁴³ and insufficient to impart significant cohesion to the nanocomposite (FIG. 1b-i&ii). PDDA, with distinct positive charges on the ammonium groups, introduces strong electrostatic interactions to the biomaterial via PDDA-LAPONITE and PDDA-gelatin binding (FIG. 1b-iii&iv), improving the integrity and cohesion. Physical appearance of the GL and pGL is shown in Figure SI in the supporting information found in Baidya et al., which is incorporated by reference.

Not only the engineered pGL biomaterial benefits from enhanced molecular and colloidal attractions, it can also be readily injected using clinically relevant catheters, e.g., 5F, as shown in FIG. 1c. This is possibly a result of reversible, noncovalent interactions of pGL building blocks, which provides shear-thinning behavior while maintaining cohesion. Continuous flow of pGL biomaterial through a 5F catheter is shown in FIG. 1c-i, and its stability after catheter injection in water is presented in FIG. 1c-ii. As the pGL biomaterial is engineered with extensive ionic interactions, structural integrity of the biomaterial may be affected by ion-rich body fluids. Therefore, to demonstrate the biomaterial cohesion in ionic solutions, it was injected in DPBS (FIG. 1e-iii). A continuous flow without any fragmentation confirmed the mechanical stability of pGL biomaterial after ionic cross-interactions.

Physical behaviors of hydrogels are mainly governed by (i) interactions between solid components and (ii) interactions of electrolyte components (e.g., water molecules and ions) with the solids⁴⁴⁻⁴⁵. In the GL biomaterial, gelatin and LAPONITE with polar and charged functional groups facilitate hydrogen bond formation and electrostatic attractions. In addition, water molecules contribute to the hydrogen bond formation between the solids, supporting the homogeneity of the biomaterial matrix. FIG. 1d-i schematically shows the composition of homogeneous GL biomaterial, with the inset showing the biomaterial at ambient conditions. Increasing the PDDA concentration increases the binding strength between the solid components, as schematically presented in FIG. 1d-ii, iii with insets showing the physical appearance of biomaterials. The charge induced enhancement in interactions is well studied in supramolecular aggregate formation⁴⁶. Electrostatic interactions with PDDA may partially replace hydrogen bonding of solids with water, increasing the cohesion. Further increase in the electrostatic interactions via increasing the PDDA concentration may cause phase separation, as shown in FIG. 1d-iii, possibly as a result of weakened hydrogen bonds that would otherwise maintain the homogeneity and solvation of macromolecules, as reported in intracellular materials⁴⁷. Enhanced dipolar interactions eventually lead to the formation of aggregates and impart thermodynamic instability to the biomaterial through phase separation⁴⁸. This is observed in the pGL at PDDA concentrations>5% w/w (FIG. 1d-iii inset). Accordingly, an optimum PDDA concentration favorably regulates the molecular/colloidal interactions in the pGL biomaterial, maintaining its homogeneity without phase separation (FIG. 1d-ii).

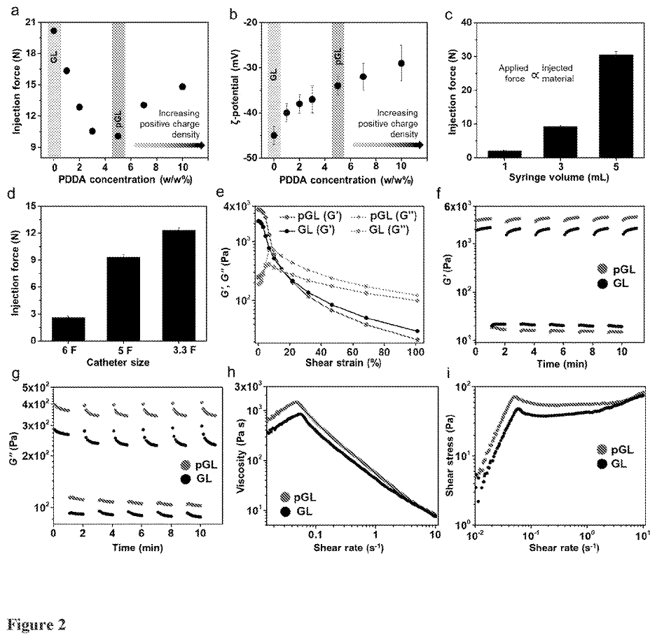

FIG. 2a shows the injectability of GL biomaterial at varying PDDA concentrations using a 5F catheter. Interestingly, the injection force was initially decreased as PDDA concentration was increased from 0 to 5% w/w, followed by an increase at higher PDDA concentrations, yielding a minimum injection force (~10 N) at a PDDA concentration of 5% w/w. This behavior may be explained by the PDDA-induced enhanced molecular interactions⁴³ and dipolar replacement of water molecules in the biomaterial, as explained in FIG. 1b,d. Mechanistically, charged PDDA might release hydrogen bonded water molecules⁴⁹ upon dipolar interactions with gelatin backbone and LAPONITE nanoplatelets, which remain locally free (unbound) and form layers between the solid constituents in the hydrogel matrix. These water layers may decrease the friction between the solid components and function as a lubricant⁴⁹. Therefore, the force required to inject the PDDA-containing GL biomaterials decreases by increasing PDDA concentrations up to about 5% w/w, while maintaining cohesion as a result of electrostatic interactions between the polymers and LAPONITE. However, extreme molecular attractions between the solids at PDDA concentration≥5% w/w caused phase separation upon excessive release of water molecules from the polymer-nanoparticle assemblies, as described in FIG. 1d-iii. Herein, unbound water molecules are permanently released from the composite biomaterial and form a separated phase. Thus, injection forces at PDDA concentration above 5% w/w increases as a result of increased solid concentration. Notably, the injection force of GL hydrogel containing PDDA across all concentrations of PDDA was lower than the GL hydrogel (FIG. 2a), which is suitable for catheter-based procedures.

FIG. 2b presents the ζ-potential of gelatin-LAPONITE aggregates at varying PDDA concentrations. By increasing the PDDA concentration, the absolute value of ζ-potential decreased, confirming the interactions of cationic PDDA with negatively charged LAPONITE-gelatin moieties. Based on the injection force and ζ-potential results, pGL biomaterial at optimum PDDA concentration (i.e., 5% w/w) was used to develop a cohesive shear-thinning hydrogel for minimally invasive procedures in all the following experiments because it did not undergo phase separation and attained the lowest injection force.

The injectability of pGL biomaterial (at 5% w/v PDDA concentration) was quantified based on various injection parameters, including injection rate, syringe volume, and catheter diameter. To demonstrate clinical applicability, catheters that are commonly used for endovascular procedures were selected for the experiments. FIG. 2c presents the injection force as a function of syringe volume while catheter diameter (5F), length (100 cm), and the flow rate (2 mL min⁻¹) maintained constant. As the syringe volume increased, a higher force was required to inject the pGL biomaterial because of the displacement of a higher biomaterial volume from the syringe to the catheter at any time. All the injection forces remained within a range that can be applied by hand⁵⁰. FIG. 2d shows the force required to inject the pGL biomaterial through catheters with varying diameters while syringe volume (3 mL), length (100 cm), and injection rate (2 mL min⁻¹) were constant. The smaller the catheter diameter the higher the injection force. In addition, the injection force increased by increasing the injection rate while catheter diameter (5F), length (100 cm), and syringe volume (3 mL) were maintained constant (Figure S2, Supporting Information found in Baidya et al., which is incorporated by reference). The injectability of pGL biomaterial through a 5F catheter was compared with GL biomaterial and DPBS (Figure S3 Supporting Information as found in Baidya et al., which is incorporated by reference). In this case, syringe volume (3 mL), length (100 cm), and injection rate (2 mL min⁻¹) were constant. Stability of pGL biomaterial (shelf life) was assessed based on injection force measurements (catheter diameter=5F, length=100 cm, syringe volume=3 mL, and injection rate=2 mL min⁻¹) at different time intervals to ensure the absence of charge induced aggregate formation and phase separation over time. The injection force of the pGL biomaterial remained nearly unchanged after 15 days and 1 month of synthesis (Figure S4, Supporting Information as found in Baidya et al., which is incorporated by reference), attesting to a decent shelf life. Within this period, coarse aggregate formation/phase separation was not observed, which would have otherwise resulted in the severe fluctuations of injection force plateau²⁹.

FIG. 2e presents the storage modulus (G′) and loss modulus (G″) of pGL and GL biomaterials versus shear strain at a constant angular frequency of ~10 rad s⁻¹. The pGL biomaterial has an improved solid-like behavior at strain <20% compared with the GL biomaterial (G′_{pGL}>G′_{GL}), and at strain>20%, the pGL attains a more liquid-like behavior (FIG. 2e). Cyclic strain recovery of pGL and GL biomaterials is presented in FIG. 2f, which shows the recovery of storage modulus for pGL and GL biomaterials with time when high (100%) and low (1%) external oscillatory strains were applied alternatively. Such a fast and reversible recovery of cohesive pGL biomaterial may be attributed to its lubricant-like behavior, originated from the locally free (unbound) water molecules between the solid constituents in the hydrogel matrix⁴⁹. Meanwhile, as a result of enhanced cohesion, the viscous nature of the pGL biomaterials (loss modulus, G″) is more pronounced than the GL (G″_{pGL}>G″_{GL}) (FIG. 2e). Both pGL and GL biomaterials underwent cyclic recovery of loss modulus as the strain alternated between high (100%) and low (1%) values over time (FIG. 2g). FIG. 2h shows the viscosity of biomaterials versus steady shear, which confirms their shear-thinning behavior. FIG. 2i presents the shear stress versus shear rate, which further demonstrates the shear-thinning behavior of pGL and GL biomaterials.

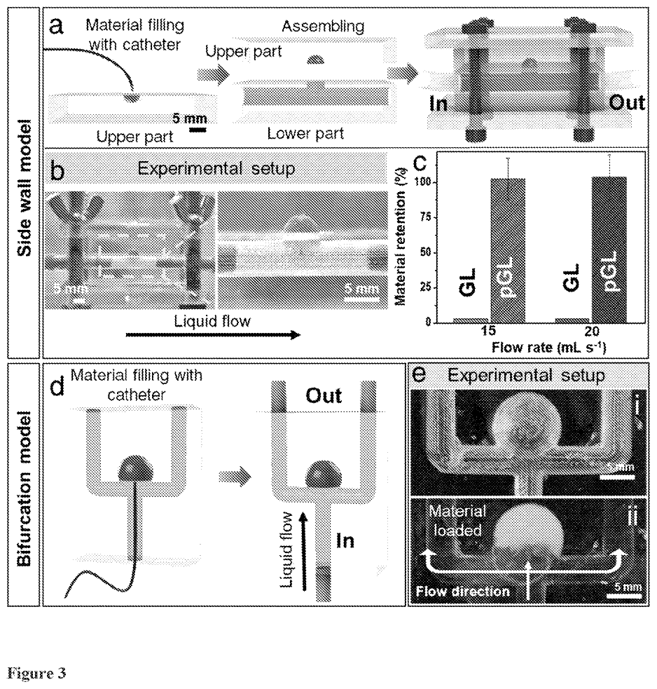

The injectability and cohesion of pGL biomaterial was tested in an in vitro cerebral aneurysm model, a type of aneurysm which is fatal in many cases^{21, 33}. Wide-neck saccular aneurysm, the most frequently observed cerebral aneurysm³⁴, was selected to be occluded with the pGL biomaterial. Two different in vitro models, namely a side-wall wide-neck aneurysm and a bifurcation aneurysm, were fabricated via 3D printing and PDMS molding. FIG. 3a schematically represents the experimental setup for the side-wall wide-neck aneurysm. In this case, a dismantlable model was fabricated to quantify the loss of material upon fragmentation during the fluid flow experiments. FIG. 3b shows the images of laboratory setup, including the loaded pGL biomaterial inside the aneurysm sac. Here, a food-grade red dye was used to enhance the contrast of biomaterial against the PDMS model. In these experiments, to assess the stability of biomaterials under fluid flow, different flow rates pertinent to cerebral blood flow were used. Considering the viscosity of blood, constant fluid (DPBS) flow rates of 15 and 20 mL s⁻¹ which are about 6.3 and 8.4 fold higher than the blood flow rate in basilar artery³⁴, respectively, were tested for 24 h. The supraphysiological flow rates compensate the shear stress for the lower viscosity of DPBS compared with blood.

During aneurysm treatment, removal of fragmented materials from the aneurysm site increases the probability of small vessel blockage, which may lead to multiple medical complications, including immediate brain death⁵¹⁻⁵². Thus, material loss was carefully observed and quantified. In all cases, the pGL biomaterial was observed to remain intact in 3 repeats. In contrast, under the same conditions, the control sample (GL biomaterial) underwent complete fragmentation and removal within an hour. To quantify material loss, samples were collected and freeze-dried until a constant mass was obtained. FIG. 3c shows the material recovery percentage at different flow rates (15 or 20 ml s⁻¹) after 24 h, which match the visual observations, attesting to the significant loss of GL biomaterial and the near-complete retention of pGL.

The stability of pGL biomaterial was further assessed in a bifurcation model, where the fluid directly contacts the material, increasing the risk of the fragmentation. FIG. 3d schematically shows the anatomy of bifurcation aneurysm model. Images of the experimental setup with or without pGL biomaterial are presented in FIG. 3e. Arrows in the picture show the fluid flow direction. The pGL biomaterial withstood both 10 and 15 mL s⁻¹ constant flow rates and remained stable for 24 h. In contrast, the control sample (GL biomaterial) was completely washed off immediately after the introduction of fluid.

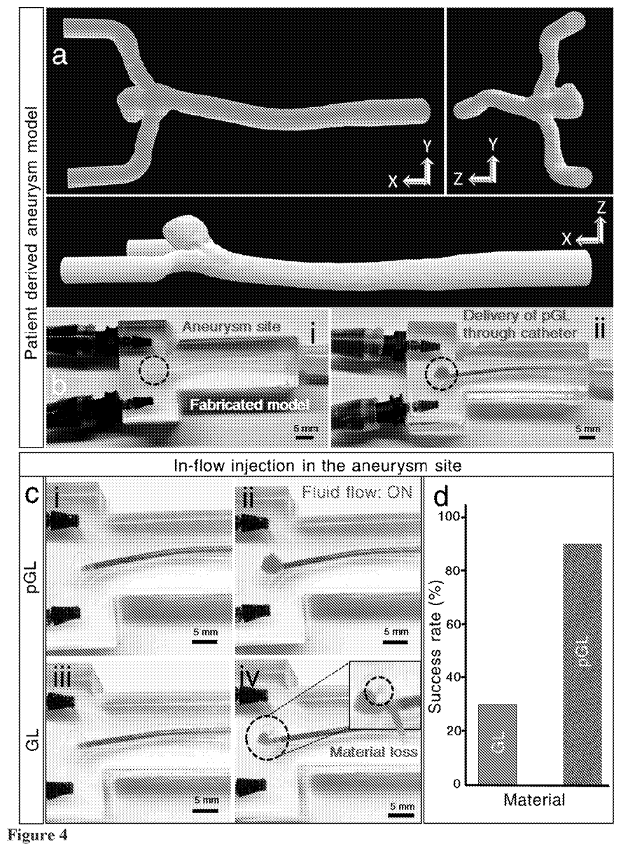

The pGL biomaterial was tested in a patient-inspired basilar tip (intracranial) aneurysm model. The model was constructed using vascular image data acquired by rotational angiography and subsequent 3D printing, followed by molding with PDMS. Details of the fabrication procedure are described in the experimental section. FIG. 4a presents the structure of patient-derived aneurysm site from different directions. FIG. 4b-i is the image of PDMS aneurysm model, fabricated using the patient data. To mimic the blood flow dynamics in the basilar artery, a carotid flow pattern³⁵ was selected. The pGL biomaterial was injected inside the basilar tip aneurysm site using a 4F catheter (FIG. 4b-ii), which is often used to treat complications in the basilar artery^{36, 53}. Given that the fragmentation of biomaterial during its injection into the aneurysm site under physiological fluid flow is one of the most important concerns, herein, the pGL biomaterial was injected in the model under a carotid fluid flow with a rate of 4 ml s⁻¹ (FIG. 4b-ii). The flow rate was ~2 fold higher than the blood flow rate in the basilar artery³⁴.

FIG. 4c shows the images of aneurysm site before (i, iii) and during (ii, iv) the delivery of pGL or GL biomaterials through a 4F catheter under the fluid flow. The GL biomaterial was immediately entrained under the flow, whereas the pGL occluded the aneurysm site consistently without undergoing fragmentation. The inset of FIG. 4c-iv shows the loss of fragmented GL biomaterial during the delivery into the in vitro model. To further demonstrate the stability of pGL biomaterial in the aneurysm site, fluid flow was continued for 3 h after removing the catheter (Figure S5, Supporting Information as found in Baidya et al., which is incorporated by reference). The success rate of pGL biomaterial delivery into the aneurysm site under the carotid fluid flow was evaluated 20 times using the patient-derived basilar tip aneurysm model, which is shown in FIG. 4d. For the GL biomaterial, in more than 70% of cases, the material was immediately fragmented during the in-flow injection into the aneurysm site under carotid fluid flow; however, the pGL biomaterial was injected and retained in the aneurysm site with a success rate of more than 90% without undergoing noticeable fragmentation.

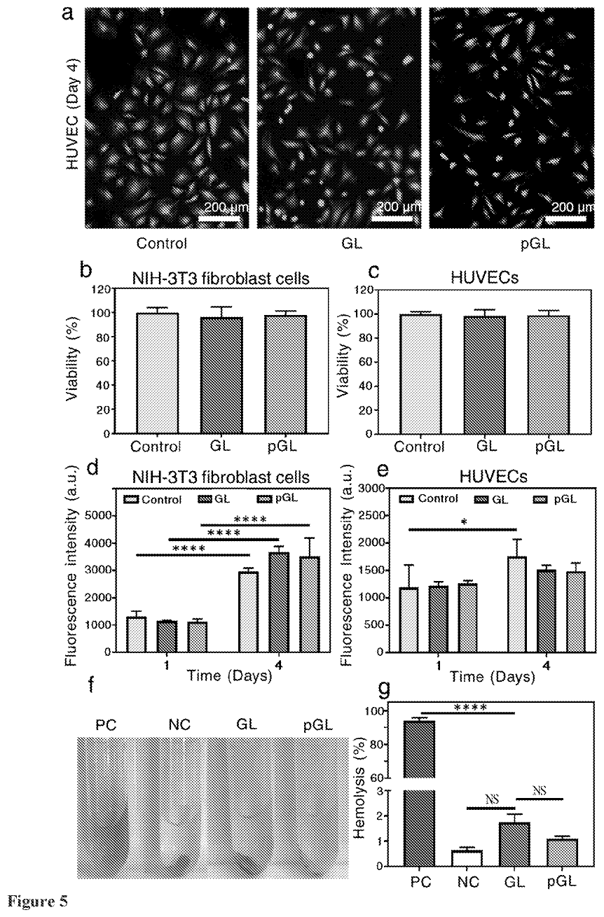

FIG. 5a shows live/dead assay images of HUVECs when exposed to biomaterials (GL or pGL) for 4 days compared with the biomaterial-free system (control). For all samples, almost all cells were stained in green (i.e., live cells), and very few dead cells (stained in red) were observed. FIGS. 5b and 5c demonstrate the quantification of live/dead assay fluorescence images for NIH/3T3 fibroblast cells and HUVECs, respectively. Approximately 96% of both cell lines remained viable after 4 days of incubation with the pGL biomaterial, similar to the GL and control samples. This indicates that the incorporation of PDDA in the GL biomaterial does not significantly affect the viability of cells. Metabolic activity of NIH/3T3 fibroblast cells and HUVECs on days 1 and 4 post incubation with pGL and GL biomaterials was assessed by the PrestoBlue™ assay and quantified in terms of fluorescence intensity, as shown in FIG. 5d and Se, respectively. The results show that over time, the metabolic activity of cells in contact with the biomaterials increases, similar to that of the control sample, which indicates that the pGL biomaterial is non-cytotoxic and does not affect cell proliferation. The metabolic activity of the NIH/3T3 fibroblasts in contact with the biomaterials at day 4 was 3-fold higher than day 1 (FIG. 5d). Similarly, the metabolic activity of HUVECs in the presence of biomaterials increased by □0.5-fold on day 4 (FIG. 5e). Therefore, the pGL biomaterial did not have any adverse effects on the cellular activities.

Biomaterial-enabled aneurysm treatment involves the direct contact of materials and blood. Therefore, the hemolytic effect of pGL biomaterial was investigated using a standard hemolysis assay in which the amount of hemoglobin released from ruptured red blood cells (RBCs) was quantified in the supernatant. The results of the hemolysis assay for the pGL and GL biomaterials with respect to the positive control (PC; Triton-X 1%) and negative control (NC; PEG 4.4%) are shown in FIG. 5f. Disruption of RBCs by the PC produced a red color in the supernatant, whereas the treatment of whole blood with the NC did not rupture the RBCs, which sedimented at the bottom of the Eppendorf tubes after centrifugation yielding a clear supernatant. To measure the percentage of biomaterial-induced hemolysis, the colorimetric quantification of supernatant was conducted, as shown in FIG. 5g. While the PC caused ~95% hemolysis, hemolysis value of the NC was below ~0.5%. Interestingly, both the GL and pGL biomaterials demonstrated significantly low hemolysis of ~2% and ~1%, respectively, which was non-significant when compared with the NC. According to the standard guidelines (ASTM E2524-08),³⁸ the permissible limit of hemolysis should be below 5%.⁵⁴ The threshold value is much higher than the value determined for the pGL (i.e. ~1%), therefore the biomaterial does not have hemolytic effects.

### CONCLUSIONS

An injectable cohesive shear-thinning biomaterial for minimally invasive procedures was developed via the polycation (PDDA)-mediated reinforcement of molecular and colloidal interactions between gelatin and LAPONITE nanoplatelets. The concentration of PDDA regulated the behavior of shear-thinning GL biomaterial. Increasing the PDDA concentration initially decreased the injection force and increased the cohesion, and as the concentration increases beyond a critical value (i.e., 5% w/w), the injection force increased, and the biomaterial eventually underwent phase separation. The optimized cohesive shear-thinning biomaterial (pGL) was able to remain intact in complex bifurcation aneurysm models under fluid flow, which was otherwise impossible to achieve using conventional GL biomaterials. To mimic the real-time intracranial aneurysm filling, the biomaterial was successfully injected and retained in the patient-derived aneurysm model under physiological fluid flow. The biocompatibility of the pGL biomaterial was confirmed using the 2D culture of NIH/3T3 fibroblast cells and HUVECs. The pGL biomaterial did not have any significant hemolytic effects. Accordingly, this novel engineered biomaterial may potentially be used for catheter-based procedures. This work addresses a long-lasting shortcoming of shear-thinning biomaterials, i.e., cohesion while maintaining injectability, which may set the stage for novel minimally invasive therapies.

### REFERENCES

(1) Zhang, K.; Feng, Q.; Fang, Z.; Gu, L.; Bian, L. Structurally Dynamic Hydrogels for Biomedical Applications: Pursuing a Fine Balance between Macroscopic Stability and Microscopic Dynamics. Chemical Reviews 2021, 121 (18), 11149-11193, DOI: 10.1021/acs.chemrev.1c00071.
(2) Lavrador, P.; Esteves, M. R.; Gaspar, V. M.; Mano, J. F. Stimuli-Responsive Nanocomposite Hydrogels for Biomedical Applications. Advanced Functional Materials 2021, 31 (8), 2005941, DOI: https://doi.org/10.1002/adfm.202005941.
(3) Uman, S.; Dhand, A.; Burdick, J. A. Recent advances in shear-thinning and self-healing hydrogels for biomedical applications. Journal of Applied Polymer Science 2020, 137 (25), 48668, DOI: https://doi.org/10.1002/app.48668.
(4) Afewerki, S.; Sheikhi, A.; Kannan, S.; Ahadian, S.; Khademhosseini, A. Gelatin-polysaccharide composite scaffolds for 3D cell culture and tissue engineering: Towards natural therapeutics. Bioeng Transl Med 2019, 4 (1), 96-115, DOI: 10.1002/btm2.10124.
(5) Davoodi, E., Montazerian, H.; Zhianmanesh, M.; Abbasgholizadeh, R.; Haghniaz, R.; Baidya, A.; Pourmohammadali, H.; Annabi, N.; Weiss, P. S.; Toyserkani, E.; Khademhosseini, A. Template-Enabled Biofabrication of Thick 3D Tissues with Patterned Perfusable Macrochannels. Advanced Healthcare Materials, 2102123, DOI: https://doi.org/10.1002/adhm.202102123.
(6) Li, J.; Mooney, D. J. Designing hydrogels for controlled drug delivery. Nature Reviews Materials 2016, 7 (12), 16071, DOI: 10.1038/natrevmats.2016.71.
(7) Oliva, N.; Conde, J.; Wang, K.; Artzi, N. Designing Hydrogels for On-Demand Therapy. Accounts of Chemical Research 2017, 50 (4), 669-679, DOI: 10.1021/acs.accounts.6b00536.
(8) Bu, Y.; Pandit, A. Cohesion mechanisms for bioadhesives. Bioactive Materials 2021, DOI: https://doi.org/10.1016/j.bioactmat.2021.11.008.
(9) Montazerian, H.; Baidya, A.; Haghniaz, R.; Davoodi, E.; Ahadian, S.; Annabi, N.; Khademhosseini, A.; Weiss, P. S. Stretchable and Bioadhesive Gelatin Methacryloyl-Based Hydrogels Enabled by in Situ Dopamine Polymerization. ACS Applied Materials & Interfaces 2021, 13 (34), 40290-40301, DOI: 10.1021/acsami.1c10048.
(10) Tavafoghi, M.; Sheikhi, A.; Tutar, R.; Jahangiry, J.; Baidya, A.; Haghniaz, R.; Khademhosseini, A. Engineering Tough, Injectable, Naturally Derived, Bioadhesive Composite Hydrogels. Advanced Healthcare Materials 2020, 9 (10), 1901722, DOI: https://doi.org/10.1002/adhm.201901722.
(11) Baghdasarian, S.; Saleh, B.; Baidya, A., Kim, H.; Ghovvati, M.; Sani, E. S.; Haghniaz, R.; Madhu, S.; Kanelli, M.; Noshadi, I.; Annabi, N. Engineering a naturally derived hemostatic sealant for sealing internal organs. Materials Today Bio 2022, 13, 100199, DOI: https://doi.org/10.1016/j.mtbio.2021.100199.
(12) Liang, K.; Bae, K. H.; Kurisawa, M. Recent advances in the design of injectable hydrogels for stem cell-based therapy. Journal of Materials Chemistry B 2019, 7 (24), 3775-3791, DOI: 10.1039/C9TB00485H.
(13) Zhu, D.; Li, Z.; Huang, K.; Caranasos, T. G.; Rossi, J. S.; Cheng, K. Minimally invasive delivery of therapeutic agents by hydrogel injection into the pericardial cavity for cardiac repair. Nature Communications 2021, 12 (1), 1412, DOI: 10.1038/s41467-021-21682-7
(14) Kim, S.; Nowicki, K. W.; Gross, B. A.; Wagner, W. R. Injectable hydrogels for vascular embolization and cell delivery: The potential for advances in cerebral aneurysm treatment. Biomaterials 2021, 277 121109, DOI: 10.1016/j.biomaterials.2021.121109.
(15) Chen, M. H.; Wang, L. L.; Chung, J. J.; Kim, Y.-H.; Atluri, P.; Burdick, J. A. Methods To Assess Shear-Thinning Hydrogels for Application As Injectable Biomaterials. ACS Biomaterials Science & Engineering 2017, 3 (12), 3146-3160, DOI: 10.1021/acsbiomaterials.7b00734.
(16) Loebel, C.; Rodell, C. B.; Chen, M. H.; Burdick, J. A. Shear-thinning and self-healing hydrogels as injectable therapeutics and for 3D-printing. Nature Protocols 2017, 12 (8), 1521-1541, DOI: 10.1038/nprot.2017.053.
(17) Avery, R. K.; Albadawi, H.; Akbari, M.; Zhang, Y. S.; Duggan, M. J.; Sahani, D. V.; Olsen, B. D.; Khademhosseini, A.; Oklu, R. An injectable shear-thinning biomaterial for endovascular embolization. Science Translational Medicine 2016, 8 (365), 365ra156-365ra156, DOI: 10.1126/scitranslmed.aah5533.
(18) Pang, Y.; Liu, J.; Moussa, Z. L.; Collins, J. E.; McDonnell, S.; Hayward, A. M.; Jajoo, K.; Langer, R.; Traverso, G. Endoscopically Injectable Shear-Thinning Hydrogels Facilitating Polyp Removal. Advanced Science 2019, 6 (19), 1901041, DOI: https://doi.org/10.1002/advs.201901041.
(19) Schroeder, G.; Edalati, M.; Tom, G.; Kuntjoro, N.; Gutin, M.; Gurian, M.; Cuniberto, E.; Hirth, E.; Martiri, A., Sposato, M. T.; Aminzadeh, S.; Eichenbaum, J.; Alizadeh, P.; Baidya, A.; Haghniaz, R.; Nasiri, R., Kaneko, N.; Mansouri, A.; Khademhosseini, A.; Sheikhi, A. Assessing the aneurysm occlusion efficacy of a shear-thinning biomaterial in a 3D-printed model. Journal of the Mechanical Behavior of Biomedical Materials 2022, 105156, DOI: https://doi.org/10.1016/j.jmbbm.2022.105156.
(20) Brilstra, E. H.; Rinkel, G. J. E.; Graaf, Y. v. d.; Rooij, W. J. J. v.; Algra, A. Treatment of Intracranial Aneurysms by Embolization with Coils. Stroke 1999, 30 (2), 470-476, DOI: doi:10.1161/01.STR.30.2.470.
(21) Hop, J. W.; Rinkel, G. J. E.; Algra, A.; Gijn, J. v. Case-Fatality Rates and Functional Outcome After Subarachnoid Hemorrhage. Stroke 1997, 28 (3), 660-664, DOI: doi:10.1161/01.STR.28.3.660.
(22) Yu, J. L.; Xu, K.; Wang, H. L.; Wang, B.; Luo, Q. Microsurgical clipping of intracranial aneurysms following unsuccessful endovascular treatment. Analysis of ten cases. Interv Neuroradiol 2010, 16 (1). 23-30, DOI: 10.1177/159101991001600103.
(23) Zhu, Y.; Zhang, H.; Zhang, Y.; Wu, H.; Wei, L.; Zhou, G.; Zhang, Y.; Deng, L.; Cheng, Y.; Li, M.; Santos, H. A.; Cui, W. Endovascular Metal Devices for the Treatment of Cerebrovascular Diseases. Advanced Materials 2019, 31 (8), 1805452, DOI: 10.1002/adma.201805452.
(24) Cruise, G. M., Shum, J. C., Plenk, H. Hydrogel-coated and platinum coils for intracranial aneurysm embolization compared in three experimental models using computerized angiographic and histologic morphometry. Journal of Materials Chemistry 2007, 17 (38), 3965-3973, DOI: 10.1039/B707177A.
(25) Zhao, J.; Lin, H.; Summers, R., Yang, M.; Cousins, B. G.; Tsui, J. Current Treatment Strategies for Intracranial Aneurysms: An Overview. Angiology 2018, 69 (1), 17-30, DOI: 10.1177/0003319717700503.
(26) Vanninen, R. L., Manninen, I. Onyx, a New Liquid Embolic Material for Peripheral Interventions: Preliminary Experience in Aneurysm, Pseudoaneurysm, and Pulmonary Arteriovenous Malformation Embolization. CardioVascular and Interventional Radiology 2007, 30 (2), 196-200, DOI: 10.1007/s00270-006-0071-2.
(27) Regine, R.; Palmieri, F.; Siero, M.; Rescigno, A.; Sica, V.; Cantarela, R.; Villari, V. Embolization of traumatic and non-traumatic peripheral vascular lesions with Onyx. Interventional Medicine and Applied Science 2015, 7 (1), 22-29, DOI: 10.1556/imas.6.2014.003.
(28) Grasso, R. F.; Cazzato, R. L., Luppi, G.; Faiella, E.; Del Vescovo, R.; Giurazza, F.; Borzomati, D.; Coppola, R.; Zobel, B. B. Pancreatic Arteriovenous Malformation Involving the Duodenum Embolized with Ethylene-Vinyl Alcohol Copolymer (Onyx). CardioVascular and Interventional Radiology 2012, 35 (4), 958-962, DOI: 10.1007/s00270-011-0272-1.
(29) Sheikhi, A.; Afewerki, S.; Oklu, R.; Gaharwar, A. K.; Khademhosseini, A. Effect of ionic strength on shear-thinning nanoclay-polymer composite hydrogels. Biomaterials Science 2018, 6 (8), 2073-2083, DOI: 10.1039/C8BM00469B.
(30) Chaparro, T. d. C., Silva, R. D.; Monteiro, I. S.; Barros-Timmons, A.; Giudici, R.; Martins dos Santos, A.; Bourgeat-Lami, E. Interaction of Cationic, Anionic, and Nonionic Macroraft Homo- and Copolymers with Laponite Clay. Langmuir 2019, 35 (35), 11512-11523, DOI: 10.1021/acs.langmuir9b01987.
(31) Chen, X.; Qiu, X.; Hou, M.; Wu, X.; Dong, Y.; Ma, Y.; Yang, L.; Wei, Y. Differences in Zwitterionic Sulfobetaine and Carboxybetaine Dextran-Based Hydrogels. Langmuir 2019, 35 (5), 1475-1482, DOI: 10.1021/acs.langmuir.8b01869.
(32) Faluk, M.; De Jesus, O. Saccular Aneurysm. In StatPearls; StatPearls Publishing Copyright © 2021, StatPearls Publishing LLC.: Treasure Island (FL), 2021.
(33) Kaneko, N., Mashiko, T.; Namba, K.; Tateshima, S.; Watanabe, E.; Kawai, K. A patient-specific intracranial aneurysm model with endothelial lining: a novel in vitro approach to bridge the gap between biology and flow dynamics. J Neurointerv Surg 2018, 10 (3), 306-309, DOI: 10.1136/neurintsurg-2017-013087.
(34) Zarrinkoob, L.; Ambarki, K.; Wåhlin, A.; Birgander, R.; Eklund, A.; Malm, J. Blood flow distribution in cerebral arteries. J Cereb Blood Flow Metab 2015, 35 (4), 648-654, DOI: 10.1038/jcbfm.2014.241.
(35) Liu, X.; Li, B.; Liu, Y.; Wu, H.; Zhang, H.; Dou, L.; Liu, C. A unique temporary collateral pathway between carotid-vertebrobasilar arteries in a carotid dissection patient. BMC Neurology 2020, 20 (1), 97, DOI: 10.1186/s12883-020-01651-1.
(36) Chapot, R.; Houdart, E.; Rogopoulos, A.; Mounayer, C.; Saint-Maurice, J.-P.; Merland, J.-J. Thromboaspiration in the Basilar Artery: Report of Two Cases. American Journal of Neuroradiology 2002, 23 (2), 282-284.
(37) Ghovvati, M.; Baghdasarian, S.; Baidya, A.; Dhal, J.; Annabi, N. Engineering a highly clastic bioadhesive for sealing soft and dynamic tissues. Journal of Biomedical Materials Research Part B: Applied Biomaterials 2022, 110 (7), 1511, DOI: https://doi.org/10.1002/jbm.b.35012.
(38) ASTM E2524-08(2013), ASTM International, West Conshohocken, PA, 2013, www.astm.org.
(39) Haghniaz, R.; Rabbani, A.; Vajhadin, F., Khan, T., Kousar, R.; Khan, A. R.; Montazerian, H., Iqbal, J.; Libanori, A.; Kim, H.-J.; Wahid, F. Anti-bacterial and wound healing-promoting effects of zinc ferrite nanoparticles. Journal of Nanobiotechnology 2021, 19 (1), 38, DOI: 10.1186/s12951-021-00776-w.
(40) Xavier, J. R.; Thakur, T., Desai, P.; Jaiswal, M. K.; Sears, N.; Cosgriff-Hernandez, E.; Kaunas, R.; Gaharwar, A. K. Bioactive Nanoengineered Hydrogels for Bone Tissue Engineering: A Growth-Factor-Free Approach. ACS Nano 2015, 9 (3), 3109-3118, DOI: 10.1021/nn507488s.
(41) Gaharwar, A. K.; Avery, R. K.; Assmann, A.; Paul, A.; Mckinley, G. H.; Khademhosseini, A.; Olsen, B. D. Shear-Thinning Nanocomposite Hydrogels for the Treatment of Hemorrhage. ACS Nano 2014, 8 (10), 9833-9842, DOI: 10.1021/nn503719n.
(42) Zapadka, K. L.; Becher, F. J.; Gomes Dos Santos, A. L.; Jackson, S. E. Factors affecting the physical stability (aggregation) of peptide therapeutics. Interface Focus 2017, 7 (6), 20170030-20170030, DOI: 10.1098/rsfs.2017.0030.
(43) Shutava, T.; Prouty, M.; Kommireddy, D.; Lvov, Y. pH Responsive Decomposable Layer-by-Layer Nanofilms and Capsules on the Basis of Tannic Acid. Macromolecules 2005, 38 (7), 2850-2858, DOI: 10.1021/ma047629x.
(44) Lim, J. Y. C., Lin, Q.; Xue, K.; Loh, X. J. Recent advances in supramolecular hydrogels for biomedical applications. Materials Today Advances 2019, 3, 100021, DOI: https://doi.org/10.1016/j.mtadv.2019.100021.
(45) Chai, Q.; Jiao, Y.; Yu, X. Hydrogels for Biomedical Applications: Their Characteristics and the Mechanisms behind Them. Gels 2017, 3 (1), 6, DOI: 10.3390/gels3010006.
(46) Datta, A.; Pati, S. K. Dipolar interactions and hydrogen bonding in supramolecular aggregates: understanding cooperative phenomena for 1st hyperpolarizability. Chemical Society Reviews 2006, 35 (12), 1305-1323, DOI: 10.1039/B605478A.
(47) Ribeiro, S. S.; Samanta, N.; Ebbinghaus, S.; Marcos, J. C. The synergic effect of water and biomolecules in intracellular phase separation. Nature Reviews Chemistry 2019, 3 (9), 552-561, DOI: 10.1038/s41570-019-0120-4.
(48) Xue, S.; Gong, R.; He, F.; Li, Y.; Wang, Y.; Tan, T.; Luo, S.-Z. Low-complexity domain of U1-70K modulates phase separation and aggregation through distinctive basic-acidic motifs. Science Advances 5 (11), eaax5349, DOI: 10.1126/sciadv.aax5349.
(49) Yang, F.; Zhao, J.; Koshut, W. J.; Watt, J.; Riboh, J. C.; Gall, K.; Wiley, B. J. A Synthetic Hydrogel Composite with the Mechanical Behavior and Durability of Cartilage. Advanced Functional Materials 2020, 30 (36), 2003451, DOI: https://doi.org/10.1002/adfm.202003451.
(50) Krayukhina, E.; Fukuhara, A.; Uchiyama, S. Assessment of the Injection Performance of a Tapered Needle for Use in Prefilled Biopharmaceutical Products. Journal of Pharmaceutical Sciences 2020, 109 (1), 515-523, DOI: https://doi.org/10.1016/j.xphs.2019.10.033.
(51) Kuriakose, D.; Xiao, Z. Pathophysiology and Treatment of Stroke: Present Status and Future Perspectives. Int J Mol Sci 2020, 21 (20), 7609, DOI: 10.3390/ijms21207609.
(52) Chugh, C. Acute Ischemic Stroke: Management Approach, Indian J Crit Care Med 2019, 23 (Suppl 2), S140-S146, DOI: 10.5005/jp-journals-10071-23192.
(53) Hanaoka, Y.; Koyama, J. I.; Fujii, Y.; Ogiwara, T., Ito, K.; Horiuchi, T. Bilateral Transradial Approach for Coil Embolization of Basilar Artery Aneurysms Associated with an Unfavorable Vertebral Artery Anatomy: Initial Experience with Role-sharing Technique. Clin Neuroradiol 2020, DOI: 10.1007/s00062-020-00971-6.
(54) Haghniaz, R.; Bhayani, K. R.; Umrani, R. D.; Paknikar, K. M. Dextran stabilized lanthanum strontium manganese oxide nanoparticles for magnetic resonance imaging. RSC Advances 2013, 3 (40), 18489-18497, DOI: 10.1039/C3RA40836A.

All publications mentioned herein (e.g. the references numerically listed above, and U.S. Patent Publication 20180104059 and the U.S. patent application Ser. No. 17/099,307) are incorporated herein by reference to disclose and describe aspects, methods and/or materials in connection with the cited publications. Certain aspects of the invention are discussed in Baidya et al., ACS Appl Mater Interfaces. 2022 Sep. 28; 14(38):42852-42863; Epub 2022 Sep. 19 (hereinafter “Baidya et al.”, the contents of which are incorporated by reference.

1. A shear-thinning biocompatible composition of matter comprising:
silicate nanoparticles or nanoplatelets;
a cationic polymer; and
gelatin.

2. The composition of claim 1, wherein the silicate nanoparticles or nanoplatelets have a median diameter of from 5 nm to 150 nm.

3. The composition of claim 2, wherein:
the silicate nanoparticles or nanoplatelets comprise from 5% to 45% (w/w) of the composition;
the cationic polymer was mixed with gelatin solution;
the cationic polymer concentration in gelatin at least 1, 2, 3, 4 or 5% (w/w);
the cationic polymer concentration in gelatin solution is less than 10, 9, 8, 7 or 6% (w/w); and/or
the gelatin comprises from 1% to 30% of the composition.

4. The composition of claim 3, wherein the silicate nanoparticles or nanoplatelets comprise not more than 9% (w/v) of the composition

5. The composition of claim 3, wherein the gelatin comprises not more than 18% (w/v) of the composition.

6. The composition of claim 4, wherein the silicate nanoparticles or nanoplatelets comprise a negative charge at physiological pH.

6. The composition of claim 5, wherein the silicate nanoparticles or nanoplatelets have a zeta potential of at least −10 mV at physiological pH.

7. The composition of claim 1, further comprising a pharmaceutical excipient selected from the group consisting of:
a preservative, a tonicity adjusting agent, a detergent, a viscosity adjusting agent, a sugar and a pH adjusting agent.

8. The composition of claim 7, further comprising a therapeutic agent.

9. The composition of claim 1, wherein the composition is disposed within a catheter.

10. A method of delivering a composition of claim I to a preselected site comprising:
disposing the composition in a vessel having a first end comprising an opening and a second end;
applying a force to the second end of the vessel, wherein the force is sufficient to liquify the composition;
delivering the composition out of the vessel through the opening and to the preselected site.

11. The method of claim 10, wherein the site is an in vivo site.

12. The method of claim 11, wherein the site is at an in vivo location where an individual has experienced trauma or injury.

13. The method of claim 10, wherein the vessel is a catheter.

14. A method of making a composition of any one of claim 1 comprising combining together silicate nanoparticles or nanoplatelets, a cationic polymer, and gelatin, so as to form a shear-thinning biocompatible composition.

15. The method of claim 14, wherein a surface property of the silicate nanoparticles, a median diameter of the silicate nanoparticles, a relative amount of silicate nanoparticles; and/or a relative amount of cationic polymer; and/or a relative amount of gelatin is selected to tune or modulate one or more properties of the composition.

16. The method of claim 15, wherein the method is selected to form a shear-thinning biocompatible composition exhibiting an injection force of less than 10 Newtons when extruded through a 5F catheter at an injection rate of 2 mL min⁻¹ under physiological conditions.

17. The method of claim 15, wherein the method is selected to form a shear-thinning biocompatible composition exhibiting a ζ-potential from −30 millivolts to −40 millivolts under physiological conditions.

18. The method of claim 15, wherein the method combines the silicate nanoparticles or nanoplatelets with a pharmaceutical excipient and/or a therapeutic agent

19. The method of claim 14, wherein:
the silicate nanoparticles or nanoplatelets are selected to have a median diameter of from 5 nm to 150 nm.
the silicate nanoparticles or nanoplatelets are selected to comprise from 5% to 45% (w/w) of the composition;
the cationic polymer concentration in gelatin is selected to comprise at least 1, 2, 3, 4 or 5% (w/w);
the cationic polymer concentration in gelatin solution is selected to comprise less than 10, 9, 8, 7 or 6% (w/w); and/or
the gelatin is selected to comprise from 1% to 30% of the composition.

20. The method of claim 19, wherein:
the silicate nanoparticles or nanoplatelets comprise not more than 9% (w/v) of the composition; and
the gelatin comprises not more than 18% (w/v) of the composition.
